(19) United States
(12) Patent Application Publication
(10) Pub. No.: US 2006/0029954 A1
(43) Pub. Date: Feb. 9, 2006

(54) Compositions and methods for identifying nucleotides in polynucleotide sequences

(75) Inventor: Kai Qin Lao, Pleasanton, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/173,887

(22) Filed: Jun. 30, 2005

Related U.S. Application Data
(60) Provisional application No. 60/584,643, filed on Jun. 30, 2004.

(51) Int. Cl. C12Q 1/68; C12P 19/34

(52) U.S. Cl. 435/6; 435/91.2

(57) ABSTRACT

The present disclosure provides methods and compositions to detect a specific nucleotide at a selected position of a polynucleotide target sequence.

This application claims benefit under 35 U.S.C. § 119(e) of U.S. Patent Application Ser. No. 60/584,643, filed Jun. 30, 2004, which is incorporated herein by reference in its entirety.

### 1. FIELD

This disclosure relates generally to compositions, methods and kits for analyzing nucleic acids, and more specifically to compositions, methods and kits for detecting and/or identifying nucleotides in polynucleotide sequences.

### 2. INTRODUCTION

Single nucleotide differences between polynucleotides, e.g., alleles, have been shown to be the cause of or a contributing factor to an individual's susceptibility to disease, disease prognosis, the efficacy of treatments and prophylaxis, and susceptibility to adverse drug reactions. As a result of the increasing amount of nucleic acid sequence information becoming available, including the entire sequence of the human genome, the identification of single nucleotide variants will play a more prominent role in medical diagnosis, tailoring treatments regimens to individual patients and genetic counseling. Identifying single nucleotides in genomes comprising billions of nucleotide base pairs is technically challenging. Furthermore, such methods should be accurate, precise and amendable to routine use in clinical settings.

There is, accordingly, a need in the art for methods of accurately and efficiently identifying single nucleotides, particularly in complex polynucleotide samples.

### 3. SUMMARY

Disclosed herein are compositions and methods for identifying a specific nucleotide at a selected position of a target sequence. In some embodiments, the target sequence is amplified by forward and reverse amplification primers, wherein one of the amplification primers has sequence suitable to amplify the target sequence having a specific nucleotide at a selected position. Therefore, in some embodiments the amplification primers produce a nucleotide specific amplicon. In some embodiments, one or more of the amplification primers may incorporate one or more sequences into the nucleotide specific amplicon suitable for detection of nucleotide specific amplicons. In some embodiments, such sequences are code sequences and/or universal sequences.

In some embodiments, the nucleotide specific amplicons are analyzed using two or more detection polynucleotides which can be modified in the presence of a nucleotide specific amplicon. In some embodiments, detection polynucleotides comprise a detection primer and a flap probe which form a substrate for the 5′-3′ nuclease activity of a polymerase when the flap probe is hybridized to the amplicon 3′ relative to the detection primer. The nuclease activity releases the flap or cleavage sequence from the probe which may be detected or, in some embodiments, may be modified prior to detection. In some embodiments, the detection polynucleotides comprise two probes which can be ligated when hybridized to the amplicon, and the ligated product can be detected, or in some embodiments, may be further modified prior to detection. In some embodiments, multiple sets of various types of detection polynucleotides can be used to detect or quantitate a plurality of nucleotide specific amplicons.

In some embodiments, at least one amplification primer can be used to amplify a target sequence having a specific nucleotide at a selected position. Therefore, in some embodiments linear amplicons can be produced and detected using one or more sets of detection polynucleotides. In some embodiments, the amplification primer incorporates into the amplicon one or more sequences suitable for providing hybridization sites for the detection polynucleotides.

In another aspect, the disclosure provides kits suitable for practicing the various embodiments of the disclosed methods. In some embodiments, a kit comprises one or more nucleotide specific amplification primers. In some embodiments, a kit may comprise one or more reverse primers suitable for synthesis of double-stranded nucleotide specific amplicons. In some embodiments, the kits can include one or more sets of detection polynucleotides suitable for detecting one or more nucleotide specific amplicons. Kits also may include one or more other reagents suitable for modifying the detection polynucleotides.

### 4. BRIEF DESCRIPTION OF THE DRAWINGS

The skilled artisan will understand that the drawings, described below, are for illustration purposes only. The drawings are not intended to limit the scope of the present disclosure in any way.

The skilled artisan will understand that the drawings, described below, are for illustration purposes only. The drawings are not intended to limit the scope of the present disclosure in any way.

FIG. 1 provides a cartoon illustrating an embodiment of the disclosed methods wherein single-nucleotide polymorphisms in allelic variants are detected using nucleotide specific amplification primers, detections primers and flap probes.

FIG. 2 provides a cartoon illustrating an embodiment of the disclosed methods wherein single-nucleotide polymorphisms in allelic variants are detected using nucleotide specific amplification primers and two ligation probes.

FIG. 3, Panels A-D provide the results of the capillary electrophoresis of control ligation amplicons used to assess the detection limit of a capillary electrophoresis detection system. Each panel shows the concentration of the ligation template. Panels E-H provide the results of the capillary electrophoresis of ligation amplicons formed by hybridization of probes to amplicons produced in a multiplex amplification using allele specific primers (ASO-1 or ASO-2). Panels E-H provide the concentration of the genomic gDNA target polynucleotide.

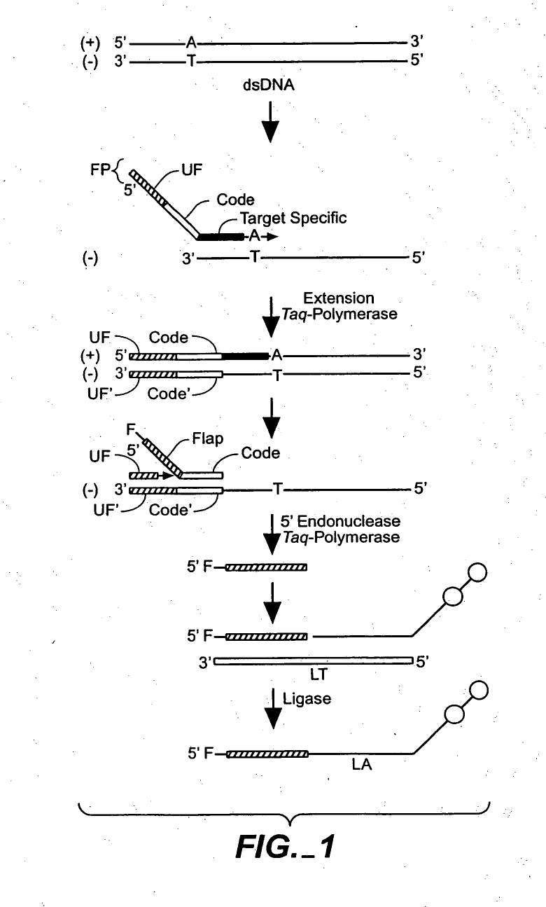

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

This disclosure provides methods, compositions and kits for detecting single nucleotides in polynucleotide sequences.

As discussed in the Summary section, in some embodiments the disclosed methods comprise amplifying a target sequence comprising a polymorphic nucleotide position with an amplification primer, a polymerase, and a mixture of deoxynucleotide triphosphates (dNTPs) suitable for DNA synthesis. By “polymorphic nucleotide position” is meant a position in a target sequence at which variable or variant nucleotides may occur. The amplification primer can be nucleotide specific in that it can be designed to amplify a target sequence having one of the possible nucleotides at the variant position. Therefore, in some embodiments the amplification product (“amplicon”) of a nucleotide specific primer may be referred to as a nucleotide specific amplicon. The amplification primer also can be designed to introduce into the amplicon a code sequence that is utilized as a surrogate or marker for the nucleotide at the variant position. In some embodiments, other sequences may be incorporated into the amplicons to facilitate analysis.

In some embodiments, at least two detection polynucleotides are hybridized to the amplicon. In some embodiments, at least one of the detection polynucleotides hybridizes to the target sequence, a code sequence or a sequence complementary thereto. When hybridized to the amplicon, at least one of the detection polynucleotides can be modified and the modified product can be detected by various methods, as described below. In some embodiments, a reporter molecule may be optionally used, for example, to monitor amplification of the target sequence and/or the modification of a detection polynucleotide. In various embodiments, the modification of a detection polynucleotide can be an isothermal reaction or the reaction may comprise thermocycling.

In some embodiments, detection polynucleotides comprise a detection primer and a “flap” probe which form a substrate for the 5′-3′ nuclease activity of a polymerase when the flap probe is hybridized to the amplicon 3′ relative to the detection primer. The nuclease activity releases a sequence (“flap” or “cleavage” sequence) from the probe that may be detected or, in some embodiments, may be modified prior to detection. In some embodiments, the detection polynucleotides comprise two probes which can be ligated when hybridized to the amplicon, and the ligated product can be detected, or in some embodiments, may be further modified prior to detection.

In some embodiments, a plurality of target sequences are simultaneously analyzed in the above-described methods in a multiplex format. “Plurality of target sequences” refers to two or more target sequences that differ by at least one nucleotide. Therefore, in some embodiments a plurality of target sequences refers to two or more related sequences (e.g., alleles, wild-type and mutant sequences, sequences of homologous genes obtained from different genera, species, subspecies, subtypes, variants, races, individuals, or sequences from one individual, for example, obtained at different time points, including but not limited to, different time points during gestation, a disease process or treatment). In some embodiments, a plurality of target sequences refers to two or more unrelated sequences (e.g., sequences from non-homologous genes) that are simultaneously analyzed by the disclosed methods.

As will be appreciated by skilled artisans, target polynucleotides may comprise one or more target sequences and may be either DNA (e.g., cDNA, genomic DNA or extrachromosomal DNA) or RNA (e.g., mRNA, rRNA or genomic RNA) in nature, and may be derived or obtained from virtually any sample or source, wherein the sample may optionally be scarce or of a limited quantity. For example, the sample may be one or a few cells collected from a crime scene or a small amount of tissue collected via biopsy. In other embodiments, the target polynucleotide may be a synthetic polynucleotide comprising nucleotide analogs or mimics, as described below, produced for purposes, such as, diagnosis, testing, or treatment.

In various non-limiting examples, the target polynucleotide may be single or double-stranded or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or a library of constructs (e.g., from a YAC, BAC or PAC library), RNA (e.g., mRNA, rRNA or vRNA) or a cDNA or a cDNA library. As known in the art, a cDNA is a single- or double-stranded DNA produced by reverse transcription of an RNA template. Therefore, some embodiments, in addition to the primers, probes, and enzymes, described above, include a reverse transcriptase and one or more “RT” primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gübler et al., 1983, Gene 25:263-269; Gtibler, 1987, Methods Enzymol., 152:330-335; Okayama et al., 1982, Mol. Cell. Biol. 2:161-170; Sellner et al., 1994, J. Virol. Method. 49:47-58; and U.S. Pat. Nos. 5,310,652, 5,322,770, and 6,300,073, these disclosures of which are incorporated herein by reference.

The target polynucleotide may include a single polynucleotide, from which one or more different target sequences of interest may be analyzed, or it may include a plurality of different polynucleotides, from which one or more different target sequences of interest may be analyzed. As will be recognized by skilled artisans, the sample or target polynucleotide may also include one or more polynucleotides comprising sequences that are not analyzed by the disclosed methods.

In some embodiments of the disclosed methods, highly complex mixtures of target sequences from highly complex mixtures of polynucleotides are analyzed in either a single-plex or multiplex format. Indeed, many embodiments are suitable for multiplex analysis of target sequences from tens, hundreds, thousands, hundreds of thousands or even millions of polynucleotides. In some embodiments, multiplex amplification methods can be used to analyze pluralities of target sequences from samples comprising cDNA libraries or total mRNA isolated or derived from biological samples, such as tissues and/or cells, wherein the cDNA or, alternatively, mRNA libraries may be quite large. For example, cDNA libraries or mRNA libraries constructed from several organisms or from several different types of tissues or organs can be amplified according to the methods described herein.

As the skilled artisan will appreciate, in multiplex embodiments multiple sets of primers and/or probes and/or reporter molecules are utilized for each target sequence to be analyzed. For example, in multiplex embodiments utilizing reporter molecules, each reporter molecule can produce a signal that is distinguishable from other reporter molecules. Therefore, in these embodiments, the number of target sequences analyzed in a multiplex format can be determined, at least in part, by the number and type of reporter molecules that may be discriminated. For example, in the embodiment, in which 5′-nuclease are probes are utilized as the reporter molecule about 2 to about 7 target sequences are analyzed in a multiplex reaction. However, in some embodiments of the disclosed methods utilizing the detection polynucleotides about 2 to about 1,000 target sequences and in some embodiments to about 7000 target sequences or more can be analyzed in a multiplex reaction (see, e.g., U.S. Patent Application Ser. Nos. 60/584,621; 60/584,665; 60/584,596, each filed Jun. 30, 2004).

The amount of target polynucleotide(s) utilized in the disclosed methods can vary widely. In many embodiments, amounts suitable for a conventional PCR and/or RT-PCR may be used. For example, the target polynucleotide(s) may be from a single cell, from tens of cells, from hundreds of cells or even more, as is well known in the art. For many embodiments, including embodiments in which the target polynucleotide is a complex cDNA library (or derived therefrom by RT of mRNA), the total amount of target polynucleotide utilized may range from about 1 pg to about 100 ng. For other embodiments, including embodiments in which the target polynucleotide(s) is obtained from a single cell, the total amount of target polynucleotide(s) may range from 1 copy (about 10 ag) to about 10⁷ copies (about 100 pg). The skilled artisan will appreciate that in some embodiments a greater number of target polynucleotides may be used.

In some embodiments, preparation of the target polynucleotide(s) for analysis may not be required. In some embodiments, the target polynucleotide(s) may be prepared for analysis using conventional sample preparation techniques. For example, target polynucleotides may be isolated from their source via differential extraction, chromatography, precipitation, electrophoresis, as is well-known in the art. Alternatively, the target sequence(s) may be amplified directly from samples, including but not limited to, cells or from lysates of tissues or cells comprising the target polynucleotide(s). Therefore, as used herein, “target sequence” also refers to an amplified target sequence. Furthermore, in some embodiments, a target sequence may be amplified prior to analysis by the disclosed methods. Examples of suitable amplification methods are well known in the art (see, e.g., U.S. Pat. Nos. 4,683,195, 4,683,202, 4,800,159, 4,965,188, 5,075,216, 5,176,995, 5,185,243, 5,386,022, 5,427,930, 5,516,663, 5,656,493, 5,679,524, 5,686,272, 5,869,252, 6,040,166, 6,197,563, 6,514,736, EP-A-0200362, EP-A-0201184 and EP-A-320308, and U.S. Patent Application Ser. No. 60/584,665, filed Jun. 30, 2004). Therefore, in some embodiments a target sequence may be amplified by a first set of amplification primers to produce amplicons that are further amplified by a second set of primers suitable for single nucleotide analysis as described below.

The number of target sequences that can be analyzed by the disclosed methods is influenced in large part by the number of different amplification primers, detection polynucleotides, and the number of different methods used to detect or discriminate the modified detection polynucleotides. In various exemplary embodiments, at least one amplification primer, at least two amplification primers, or at least three amplification primers or more may be used to amplify a target sequence. By “primer” herein is meant a polynucleotide capable of hybridizing or annealing to a template polynucleotide to form a substrate for a polymerase (e.g., DNA-dependent DNA polymerases, RNA-dependent DNA polymerase (reverse transcriptase)). When a primer is hybridized to its template, a polymerase is capable of initiating synthesis of a nascent polynucleotide strand in a template directed manner at the 3′ terminus of the primer. Therefore, in various embodiments, a primer can be an amplification primer and/or a reverse transcription primer. In some embodiments, a primer may be a detection polynucleotide, as described below. By “annealing” or “hybridizing” is meant base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing.

In various embodiments, an amplification primer may be an “exponential primer” and/or a “linear primer.” By “exponential primer” and “exponential amplification primer” herein are meant a primer suitable for exponential amplification of a polynucleotide sequence. In exponential target sequence amplification, the product of each amplification cycle is an amplicon that is a suitable template for subsequent amplification cycles. Therefore, as known in the art, exponential amplification generally utilizes at least two or paired exponential primers. For example, the exponential amplification of a target sequence by PCR generally utilizes a pair of “forward” and “reverse” primers. Therefore, the skilled artisan is aware that the suitability of a primer for exponential amplification depends, in part, on the presence of a second suitable primer. The forward and reverse primers hybridize to a target sequence in opposite orientations to produce complementary DNA strands to form double-stranded amplicons that serve as templates for further rounds of amplification. By “linear primer” and “linear amplification primer” herein are meant a primer suitable to linearly amplify a polynucleotide sequence. In linear target sequence amplification, the product of each amplification cycle is not suitable for subsequent amplification cycles. For example, the linear amplification of a target sequence generally produces a single-stranded amplicon that does not hybridize to the linear primer and, therefore, is not a suitable template for subsequent amplification cycles. As a result, in some embodiments, linear amplicons accumulate at a rate proportional to the number of templates. In some embodiments, the use of exponential and linear amplification reactions may be used to quantitate the number of target sequences (see, e.g., U.S. Patent Application Ser. No. 60/584,621, filed Jun. 30, 2004.

The amplification primers may be target sequence-specific or may be designed to hybridize to sequences that flank a target sequence to be amplified. In some embodiments, an amplification primer may be designed to hybridize to and specifically amplify a target sequence having a specific nucleotide at a selected nucleotide position. Therefore, a “nucleotide specific” amplification primer may be designed to be inefficiently extended by the action of a polymerase when hybridized to a target sequence having an “incorrect” or “inappropriate” nucleotide at the selected position. To be inefficiently extended when hybridized to a target sequence having an inappropriate nucleotide, in some embodiments, an amplification primer may be designed not to base pair with the target polynucleotide in a region at or near the amplification primer's 3′ terminus. Therefore, an amplification primer may not provide a suitable substrate for a polymerase resulting in inefficient extension and amplification. (see, e.g., Kornberg, DNA Replication (W.H. Freeman & Co. 1980)). In a non-limiting example, the 3′ terminal nucleotide of an amplification primer may be designed to amplify a target sequence having each an A, G, T or C, at a selected position of the target sequence. Thus, the actual nucleotide sequences of each primer may depend upon the specific nucleotide of the target sequence to be analyzed and the target polynucleotide, which will be apparent to those of skill in the art. Methods for designing primers suitable for amplifying target sequences of interest are well-known (see, e.g., Dieffenbach et al., General Concepts for PCR Primer Design, in PCR Primer, A Laboratory Manual, Dieffenbach, C. W, and Dveksler, G. S., Ed., Cold Spring Harbor Laboratory Press, New York, 1995, 133-155; Innis, M. A. et al. Optimization of PCRs, in PCR protocols, A Guide to Methods and Applications, Innis, M. A., Gelfand, D. H., Sninsky, J. J., and White, T. J., Ed., CRC Press, London, 1994, 5-11; Sharrocks, et al. The design of primers for PCR, in PCR Technology, Current Innovations, Griffin, H. G., and Griffin, A. M, Ed., CRC Press, London, 1994, 5-11; Suggs et al., Using Purified Genes, in ICN-UCLA Symp. Developmental Biology, Vol. 23, Brown, D. D. Ed., Academic Press, New York, 1981, 683; Kwok et al. Effects of primer-template mismatches on the polymerase chain reaction: Human Immunodeficiency Virus 1 model studies. Nucleic Acids Res. 18:999-1005, 1990; Compton T (1990). Degenerate primers for DNA amplification. pp. 39-45 in: PCR Protocols (Innis, Gelfand, Sninsky and White, eds.); Academic Press, NY; Fuqua et al. (1990). BioTechniques 9(2):206-211; Gelfand et al., 1990, Thermostable DNA polymerases. pp. 129-141 in: PCR Protocols (Innis, Gelfand, Sninsky and White, eds.); Academic Press, NY; Innis et al., 1990, Optimization of PCRs. pp. 3-12 in: PCR Protocols (Innis, Gelfand, Sninsky and White, eds.); Academic Press, NY; Krawetz et al., 1989, Nucleic Acids Research 17(2):819; Rybicki et al., 1990, Journal of General Virology 71:2519-2526; Rychlik et al., 1990, Nucleic Acids Research 18(21):6409-6412; Sarkar et al., 1990, Nucleic Acids Research 18(24):7465; Smith et al., 1990, 9/90(5):16-17; Thweatt et al. 1990, Analytical Biochemistry 190:314-316; Wu et al., 1991, DNA and Cell Biology 10(3):233-238; Yap et al., 1991, Nucleic Acids Research 19(7):1713, which provide examples demonstrating how particular primer pairs may be designed.)

Generally, each amplification primer should be sufficiently long to prime template-directed synthesis under the conditions of the disclosed methods. The exact lengths of the primers may depend on many factors, including but not limited to, the desired hybridization temperature between the primers and template polynucleotides, the complexity of the different target polynucleotide sequences to be amplified, the salt concentration, ionic strength, pH and other buffer conditions, and the sequences of the primers and target polynucleotides. The ability to select lengths and sequences of primers suitable for particular applications is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning. A Laboratory Manual 10.1-10.10 (3d. ed. Cold Spring Harbor Laboratory Press)). In some embodiments, the primers contain from about 15 to about 35 nucleotides that are suitable for hybridizing to a target sequence and form a substrate suitable for DNA synthesis, although the primers may contain more or fewer nucleotides. Shorter primers generally require lower temperatures to form sufficiently stable hybrid complexes with target sequences. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In some embodiments, in which thermocycling occurs, the amplification primers should be designed to have a melting temperature (“Tₘ”) in the range of about 60-75° C. Melting temperatures in this range tend to insure that the primers remain annealed or hybridized to the target polynucleotide at the initiation of primer extension. The actual temperature used for a primer extension reaction may depend upon, among other factors, the concentration of the various primers and the types of detection polynucleotides employed, as described below, and methods used to detect the modified detection polynucleotides. For amplifications carried out with a thermostable polymerase such as Taq DNA polymerase, the amplification primers can be designed to have a Tₘ in the range of about 60 to about 78° C. The melting temperatures of the different amplification primers can be different; however, in an alternative embodiment they can all be approximately the same, i.e., the Tₘ of each amplification primer can be within a range of about 5° C. or less. The Tₘs of various primers can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)). Alternatively, the Tₘ of a amplification primer can be calculated. Numerous references and aids for calculating Tₘs of primers are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10.1-10.10 (3d. ed. Cold Spring Harbor Laboratory Press)); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference. Any of these methods can be used to determine a Tₘ of a primer.

As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). Accordingly, in some embodiments, another factor to consider, in addition to those described above, when designing any primer is the structure of the primer and target polynucleotide. For example, in the embodiment in which an RNA polynucleotide is reverse transcribed to produce a cDNA, the determination of the suitability of a DNA primer for the reverse transcription reaction can include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

The concentration of an amplification primer may vary widely and in various embodiments, may be limiting or non-limiting. “Limiting concentration” refers to a concentration of a reagent, such as, an amplification primer, that determines the rate at which a reaction may proceed and/or the time point at which a reaction terminates. Conversely, “non-limiting concentration” refers to a concentration of a reagent at the point a reaction initiates that may not determine the rate at which the reaction may proceed and/or the time point at which the reaction terminates. A skilled artisan will appreciate, however, that in some embodiments a reagent at a non-limiting concentration may become limiting as the reagent is consumed during the course of the reaction. In some embodiments, a limiting concentration of an amplification primer terminates the amplification reaction before it reaches a plateau. In some embodiments, the concentration of an amplification primer can be adjusted so that a selected number of amplicons are generated. Determining the appropriate concentration of one or more amplification primers is within the abilities of the skilled artisan. Examples of factors to be considered include but are not limited to the quantity of target sequence, the relative amount of each target polynucleotide sequence to be amplified, the number of different target polynucleotides sequences amplified in a single reaction (i.e., multiplex or single-plex), the sensitivity of the detection system, and the degree of accuracy desired. In various exemplary embodiments, a limiting concentration of an amplification primer is less than about 50 nM, less than about 40 nM, less than about 30 nM, less than about 20 nM, or less than about 10 nM. In some embodiment, a limiting concentration of an amplification limiting primer is about 10 nM to about 30 nM. Exemplary embodiments of non-limiting concentrations include, a concentration of at least about 100 nM, at least about 500 nM, at least about 1 μM or even greater.

The target specific sequences of amplification primers used in the disclosed methods are designed to be substantially complementary to regions of the target polynucleotides. By “substantially complementary” herein is meant that the sequences of the amplification primers include enough complementarity to hybridize to the target polynucleotides at the concentration and under the temperature and conditions employed and to be capable of being extended by a DNA polymerase. Although in some embodiments the sequences of the primers may be completely complementary to a target polynucleotide, in other embodiments it may be desirable to include one or more nucleotides of mismatch or non-complementarity, as is well known in the art. By “regions of mismatch” and “non-complementarity” are meant a least one nucleotide of a polynucleotide sequence that is not suitable for base-pairing with another polynucleotide sequence. Therefore, the term “region of mismatch” is used when comparing sequences, such as, a primer sequence and another primer sequence; a primer sequence and a target sequence; a probe sequence and a target sequence; a primer sequence and an amplicon sequence; and the like. Therefore, a “region of mismatch” includes a “region of sequence diversity.” As the skilled artisan will appreciation, a region of mismatch between an amplification primer and a target sequence may be incorporated into the resulting amplicons. In some embodiments, regions of mismatch may be incorporated into amplicons to provide useful cites for hybridizing to detection polynucleotides, as described below.

In some embodiments, an amplification primer sequence that is a region of mismatch in comparison to a target sequence is substantially unique to that primer. Therefore, in some embodiments, a region of mismatch between an amplification primer and a target sequence is a code sequence. By “code sequence” is meant a sequence of continuous nucleotides that is substantially unique. “Substantially unique” refers to a sequence suitable to identify or distinguish the polynucleotide comprising the code sequence. In some embodiments, code sequences may be used to identify the amplification product of a specific primer and/or to identify the product of a modified detection polynucleotide. Therefore, the skilled artisan will appreciate that in some embodiments code sequences may be used for the manipulation, detection and/or analysis of polynucleotides.

In some embodiments, a region of mismatch between an amplification primer and a target sequence is a sequence that is shared by more than one amplification primer. In some embodiments, the shared sequence may also be a sequence of a probe. In non-limiting exemplary embodiments, a “shared sequence” may be common to each forward primer or each reverse primer. Thus, “forward universal sequence” and “reverse universal sequence” refers to a primer sequence of continuous nucleotides that is a region of diversity in comparison to a target sequence that is shared by each forward or reverse primer, respectively.

Determining the number, type, length, and composition of the various regions of an amplification primer and their distribution or commonality among the various polynucleotides employed in the disclosed methods are within the capabilities of the ordinary skilled artisan. Amplification primers and methods for incorporating various types of sequences into amplification primers and amplicons derived therefrom are known in the art (see, e.g., U.S. Pat. Nos. 5,314,809, 5,853,989, 5,882,856, 6,090,552, 6,355,431, 6,617,138, 6,630,329, 6,635,419, 6,670,130, 6,670,161 and Weighardt et al., 1993, PCR Methods and App. 3:77 and, the disclosures of which are incorporated by reference).

In some embodiments, nucleotide specific amplification primers may be used to analyze or determine the possible nucleotides, e.g., variant nucleotides, that may occupy one or more positions in a target polynucleotide sequence. In some embodiments, a nucleotide specific primer amplifies a target sequence having a specific nucleotide at a selected position to produce “nucleotide specific amplicons” which may be detected, as described below. In embodiments, including but not limited to multiplex amplification reactions, a nucleotide specific primer may comprise a code sequence, suitable for facilitating detection and discrimination of each amplicon and, therefore, each variant nucleotide. In some embodiments, a nucleotide specific primer may comprise a universal sequence suitable to hybridize to a detection polynucleotide, as described below. In some embodiments, a detection polynucleotide may hybridize to the universal sequence or a sequence complementary thereto. In various non-limiting examples, variant nucleotides that are detected may represent a naturally occurring variant (e.g., an allelic variant, a single-nucleotide polymorphism (SNP)), a mutation resulting from the application of positive or negative selection pressure on a cell or organism (e.g., drug resistant variant or factor-dependent variant), a somatic mutation (e.g., mutations occurring in genes encoding antibodies during the maturation of an immune response), a mutation produced by an in vitro mutagenesis technique, including but not limited to, site specific mutagenesis, cassette or PCR mutagenesis, scanning mutagenesis, gene shuffling, recursive sequence recombination, and the like.

To detect and analyze the nucleotide specific amplicons produced using a nucleotide specific amplification primer, in some embodiments, the amplicons are hybridized to at least two detection polynucleotides. When hybridized to the amplicon, the detection polynucleotides form a substrate which is modified to form a detectable product. In various exemplary embodiments, “modified” refers to cleavage, extension, ligation, and/or labeling of a detection polynucleotide. The skilled artisan will appreciate that in some embodiments, including but not limited to multiplex reactions, each pair of detection polynucleotides may comprise a substantially unique substrate which is modified to form a substantially unique product. Therefore, in some embodiments, each product detected may be traced to one of the possible nucleotides at the selected position in the target sequence.

In some embodiments, the detection polynucleotides comprise a “flap” probe and a detection primer. “Flap probe” or “cleavage probe” refers to a probe comprising at least two domains or regions. One probe domain comprises a nucleobase sequence suitable for hybridizing to a target polynucleotide and, therefore, is substantially complementary to a target polynucleotide. Another probe domain comprises a nucleobase sequence that is not suitable for hybridizing to a target polynucleotide. Therefore, when a flap probe hybridizes to its target polynucleotide, one domain of the probe forms one strand of a double-stranded nucleic acid and another domain forms a single-stranded region, i.e., a “flap” or “cleavage” sequence. The skilled artisan will appreciate that the definition of flap probe provided herein, differs from a “conventional probe” which does not provide a “flap” or “cleavage” sequence suitable for release by the 5′-3′ nuclease activity of a polymerase when hybridized to its complementary sequence, and wherein the released flap or cleavage sequence is suitable for detection as described below. In various embodiments, the target specific and flap sequences may be in any orientation. Therefore, in some embodiments, the flap sequence is 5′ relative to the target specific sequence, and, in some embodiments, the flap is 3′ relative to the target specific sequence.

In some embodiments, thermocycling is employed to form additional substrates for the nuclease activity of the polymerase after cleavage of the hybridized flap probes. In some embodiments, a substrate for the 5′-3′ nuclease activity may be formed by the hybridization of the flap probe and detection primer under conditions suitable for extension of the detection primer by the polymerase.

In various exemplary embodiments, the target specific sequences of the flap probes may be designed to be substantially complementary to the target sequence, to a region that flanks the target sequence, including but not limited to, a universal sequence, a code sequence, or sequences complementary thereto. The actual nucleobases that comprise each hybridization sequence may depend upon the complexity of the target polynucleotides being analyzed, and the number of type of sequences incorporated into the amplicons, which will be apparent to those of skill in the art. Generally, the parameters described above in the design of amplification primers are applicable to the design of the target specific sequences of the flap probes.

In contrast to the target specific sequences, the flap or cleavage sequences can be designed to be substantially non-complementary to the target polynucleotides. Therefore, the flap sequences are regions of mismatch relative to the target polynucleotides. The actual nucleobases that comprise each flap sequence may depend upon the number and type of target sequences and target polynucleotides to be analyzed, the assay conditions (e.g., temperature, pH, ionic strength, etc.), and the extent to which each flap sequence may be discriminated. Therefore, in some embodiments, each flap sequence may be substantially unique or provide a code sequence. In some embodiments, a flap sequence may not be substantially unique and, therefore, may have statistically significant sequence homology to the flap sequence of another flap probe. Therefore, in some embodiments, two or more flap sequences may be identical in length and/or composition. Embodiments in which such flap sequences find use include, but are not limited to, assays in which a sample is screened for the presence or absence of one or more target polynucleotides having the same or different nucleotides at a selected position. In such embodiments, discrimination of the released flap sequences and, therefore, discrimination of the various target polynucleotides is generally not desired.

In some embodiments, wherein the flap sequence is released by the 5′-3′ nuclease activity of a polymerase, the probe is hybridized to a target sequence 3′ relative to a detection primer. By “detection primer” herein is meant a polynucleotide capable of hybridizing or annealing to a template polynucleotide to form a substrate for a polymerase at a position that is 5′ relative to a flap probe. Therefore, when a detection primer is hybridized to a target polynucleotide at a position 5′ relative to a flap probe, a substrate for the 5′-3′ nuclease activity of a polymerase is formed. In some embodiments, a detection primer may also function as an amplification primer as described above. In some embodiments, a detection primer and an amplification primer are different polynucleotides. Therefore, in some embodiments, a detection primer may hybridize to a universal sequence, a code sequence or sequences complementary thereto. Generally, the parameters described above in the design of amplification primers are applicable to the design of the detection primers.

Non-limiting examples of polymerases with 5′-3′ nuclease activity include, but are not limited to, AmpliTaq® GOLD, AmpliTaq® FS and AmpliTaq® DNA polymerase (Applied Biosystems, Foster City, Calif.), E. coli DNA polymerase I (New England Biolabs, Beverly, Mass.), rBst DNA Polymerase (Epicenter®, Madison, Wis.), and Tfl DNA polymerase (Promega Corp., Madison, Wis.). The nuclease activity of the polymerase and its capability to release the flap sequence is, at least in part, influenced by the distance between the 3′ terminus of the detection primer and the most 5′ nucleobase of the probe that is hybridized to the target sequence. Therefore, in some embodiments, extension of the primer by the polymerase may not be required for the flap probe to be released. For example, if the 3′ terminus of the primer is at least within about 20 nucleobases of the 5′ hybridized nucleobase of the probe, primer extension and, therefore, target sequence amplification may not be required for release of the flap sequence. In embodiments wherein the distance between the primer and probe is greater than about 20 nucleobases, extension of the detection primer may be required for release of the flap sequence from a probe. Therefore, in some embodiments, the primer may be extended such that its 3′ terminus is within at least about 20 nucleobases of the hybridized probe. In some embodiments, the primer may be extended to amplify the target sequence. Therefore, in some embodiments, release of the flap sequence may occur during amplification of the target sequence.

Once released, the flap sequences may be detected by various techniques as known in the art. In some embodiments, a released flap sequence may be detected directly without modification. Therefore, in some embodiments the released flap sequence can be detected in the form in which is it released. In various non-limiting examples, a released flap sequence may be directly detected by capillary electrophoresis (see, e.g., U.S. Pat. Nos. RE37,941, 6,372,106, 6,372,484, 6,387,234, 6,387,236, 6,402,918, 6,402,919, 6,432,651, 6,462,816, 6,475,361, 6,476,118, 6,485,626, 6,531,041, 6,544,396, 6,576,105, 6,592,733, 6,596,140, 6,613,212, 6,635,164, 6,706,162) or by array-based assays (see, e.g., U.S. Pat. Nos. 5,405,783, 5,445,934, 5,510,270, 5,547,839, 6,232,062, 6,221,583, 6,309,822, 6,344,316, 6,355,431, 6,355,432, 6,368,799, 6,396,995, 6,410,229, 6,440,667, 6,576,425, 6,576,424 6,600,031, 6,632,605, 6,646,243, 6,495,323, 6,667,394, 6,670,122, 6,686,150). However, the skilled artisan will appreciate that, in some embodiments, a modified flap sequence, as described below, may be detected by methods suitable for detecting an unmodified flap sequence.

In some embodiments, a released flap sequence may be modified prior to detection. For example, in some embodiments, the released flap sequence may comprise the ligand of a binding partner or an anti-ligand. Therefore, in some embodiments, a flap sequence is modified by the binding of the binding partner to the ligand. Thus, “ligand,” “binding partner” and “anti-ligand” as used herein refer to molecules that specifically interact with each other. “Specifically interact” refers to binding that is substantially distinctive and restricted, and sufficient to be sustained under conditions that inhibit non-specific binding. Non-limiting examples of ligand binding include but are not limited to antigen-antibody binding (including single-chain antibodies and antibody fragments (e.g. Fab, Fab′, F(ab′)₂, Fv)), hormone-receptor binding, neurotransmitter-receptor binding, polymerase-promoter binding, substrate-enzyme binding, allosteric effector-enzyme binding, biotin-streptavidin binding, digoxin-anti-digoxin binding, carbohydrate-lectin binding, or a molecule that donates or accepts a pair of electrons to form a coordinate covalent bond with the central metal atom of a coordination complex. In various exemplary embodiments, the dissociation constant of the ligand/anti-ligand complex is less than about 10⁻⁴-10⁻⁹ M⁻¹, less than about 10⁻⁵-10⁻⁹ M⁻¹ or less than about 10⁻⁷-10⁻⁹ M⁻¹. In some embodiments, a ligand and/or binding partner comprise one or more detectable moieties, asdescribed below.

In some embodiments, a released flap sequence is modified by the action of one or more enzymes. Non-limiting examples of enzymes suitable for modifying a released flap sequence include polymerases (e.g., DNA-directed DNA polymerases, RNA-directed DNA polymerases, terminal transferases, thermostable polymerases (e.g., Taq, Pfu, Vent), reverse transcriptases, Klenow fragment, T4 DNA polymerase, T7 DNA polymerase, ligases (e.g., thermostable ligases, T4 DNA ligase), polynucleotide kinases, phosphatases (e.g., bacterial alkaline phosphatase, calf intestinal alkaline phosphatase, shrimp alkaline phosphatase), endonucleases (e.g., restriction endonucleases I-III), and exonucleases (e.g., exonucleases I-III, mung bean nuclease, BAL31 nuclease, S1 nuclease). Therefore, in various exemplary embodiments, a released flap sequence may be modified by the addition or removal of nucleotides or phosphate groups, by ligation to another polynucleotide, by cleavage of the flap sequence, by the addition or removal of a moiety (e.g., a ligand or a moiety suitable for producing a detectable signal, as described below), or by amplification of the released flap sequence (e.g., by PCR, LCR, LDR, OLA). In some embodiments, a released flap sequence may be suitable to initiate a coupled amplification reaction (see, e.g., U.S. Patent Application Ser. No. 60/584,665. filed Jun. 30, 2004).

The skilled artisan will appreciate that in some embodiments modification or detection of a released flap sequence may include hybridizing the released flap sequence to another polynucleotide, such as, a primer, a probe, or template (e.g., a polymerization template or ligation template). When hybridized to another polynucleotide, the released flap sequence, in various embodiments, may itself function as a probe, template, primer and/or a substrate (e.g., a ligation partner, as described below). Therefore, in some embodiments, a flap sequence may be designed to be substantially complementary to a polynucleotide that is used in methods of detecting the released flap sequence. In some embodiments, wherein a released flap sequence is ligated to a ligation partner, the flap sequence and ligation partner are hybridized to a ligation template under conditions suitable for a ligase to form a covalent bond between the 3′-hydroxyl of one sequence and the 5′-phosphate of the other sequence. Thus, in some embodiments, the resulting “ligation product” may be formed by ligating the flap sequence to the 3′ or 5′ terminus of the ligation partner. In some embodiments, the conditions suitable for ligation may include thermocycling in the presence of a thermostable ligase and therefore, in some embodiments, a ligation partner can be a ligation amplicon. In some embodiments, the flap and ligation partner sequences, when hybridized to the ligation template, may be separated by a gap of at least one nucleotide and, therefore, are not suitable for ligation. Therefore, in some embodiments, the sequence hybridized to the ligation template 5′ relative to the other sequence may be extended by the action of a polymerase. In some embodiments, a gap between the hybridized flap and ligation partner sequences may be filled-in by hybridizing one or more ligation partners to the ligation template.

Generally, each released flap sequence should be sufficiently long and comprise a sequence sufficient for its detection or quantitation by the method selected by the practitioner. Similarly, the polynucleotides employed to detect or quantitate a released flap sequence also should be sufficiently long and comprise a sequence suitable for detecting or modifying the released flap sequence. Factors to be considered in selecting the length and composition of a flap sequence and the polynucleotides employed in its detection or modification include but are not limited to, the method of detection, the efficiency of a reaction selected to modify the released flap sequence, the number of types of polynucleotides employed to detect the released flap sequences, the conditions under which the flap sequence is released, the presence or absence of moieties on the released flap sequence (e.g., ligands or detectable moieties), the complexity of the different target polynucleotides to be analyzed, the complexity of the different flap sequences, and the reaction conditions (e.g., temperature, salt concentration, ionic strength, pH, and the like). The ability to design flap sequences and polynucleotides of suitable length and composition is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10.10 (3d. ed. Cold Spring Harbor Laboratory Press)). However, generally, flap sequences comprise from about 15 to about 35 nucleotides, although in some embodiments the flap sequences may contain more or fewer nucleotides/nucleobases. Furthermore, polynucleotides employed to detect or modify a released flap sequence may be shorter or longer than the flap sequence. As described above, the capability of sequences to anneal can be determined by the melting temperature (“Tₘ”) of the hybrid complex. Thus, the factors described herein in the design of target specific sequences suitable for hybridizing to a target polynucleotide are, in some embodiments, applicable to the design of flap sequences and polynucleotides used for their detection or modification.

In some embodiments, the detection polynucleotides comprise at least two ligation probes. Therefore, in some embodiments, the ligation probes hybridize to a target sequence, e.g., a nucleotide specific amplicon, and can be modified by being joined to form a single polynucleotide (e.g., ligation product or ligation amplicon). In some embodiments, at least one ligation probe hybridizes to a sequence that is substantially unique to the nucleotide specific amplicon. Therefore, the ligation product may be traced to the nucleotide specific amplicon and the target sequence that was amplified. In various exemplary embodiments, the substantially unique sequence may be the target sequence, a code sequence, or sequences complementary thereto. Therefore, any one or more other ligation probes may be hybridized to a substantially unique nucleotide specific amplicon sequence or a sequence shared by other amplicons. In some embodiments a first ligation probe hybridizes to a substantially unique sequence, e.g., a code sequence and at least one other ligation probe hybridizes to a sequence shared with at least one other amplicon, e.g., a universal sequence.

In some embodiments, the ligation probes hybridize to a nucleotide specific amplicon to form a substrate for a ligase under conditions suitable for a ligase to form a covalent bond between the 3′ hydroxyl of one ligation probe and the 5′ phosphate of another ligation probe. In some embodiments, the conditions suitable for ligation may include thermocycling in the presence of a thermostable ligase. In some embodiments, one or more ligation probes, when hybridized to the nucleotide specific amplicon, may be separated by a gap of at least one nucleotide and, therefore, are not suitable for ligation. Therefore, as described above for the released flap sequences, in some embodiments, the sequences hybridized to the ligation template 5′ relative to the other sequence may be extended by the action of a polymerase. In some embodiments, a gap between the hybridized sequences may be filled-in using one or more additional ligation probes. The skilled artisan will appreciate, that the ligation amplicon may be detected by any one or more of the methods described above for the released flap sequences.

In the exemplary embodiments, described above, the two detection polynucleotides are hybridized to one strand of a nucleotide specific amplicon. Thus, in some embodiments the detection polynucleotides may be employed in a linear amplification reaction. The skilled artisan will appreciate that in some embodiments the detection polynucleotides may be employed in an exponential amplification reaction. In one non-limiting example, the forward and reverse primers for the production of a double-stranded, nucleic acid specific amplicon each may incorporate a code sequence and universal sequence into both strands of the amplicon. Therefore, in some embodiments two detection polynucleotides are hybridized to each strand of the nucleotide specific amplicon, which further increases the sensitivity of the detection method. Thus in various embodiments, a nucleotide specific amplicon may contain virtually any number of sequences suitable for hybridizing to the detection polynucleotides, as disclosed herein.

The various polynucleotides described herein may be of any chemical composition that is suitable for the polynucleotide to carry out its intended function. Thus, in one non-limiting example, a flap probe may be of any chemical composition suitable for hybridizing to a target polynucleotide and for providing a flap sequence suitable for release by the 5′-3′ nuclease activity of a polymerase under the conditions of the disclosed methods. Therefore, in some embodiments, a flap probe may comprise nucleobases that are substantially resistant to the 5′-3′ nuclease activity of a polymerase with the exception of a sequence within the probe that is to be cleaved by the nuclease activity. In another non-limiting example, a primer may be of any chemical composition suitable for hybridizing to a template and for providing a substrate for template directed primer extension by the action of a polymerase. In another non-limiting example, a ligation probe may be of any chemical composition suitable for hybridizing to a nucleotide specific amplicon and being ligated by the a thermostable ligase. Determining the types of nucleobase polymers suitable for the function of each polynucleotide is within the abilities of the skilled artisan.

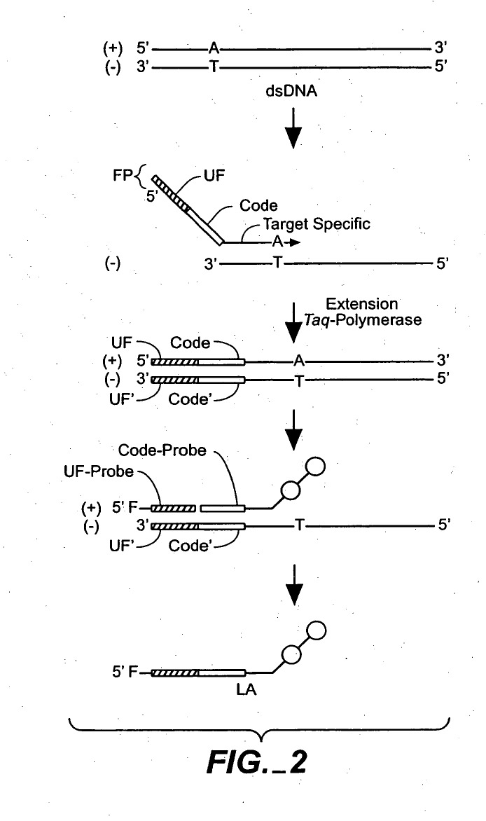

Therefore, by “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (U.S. Pat. No. 6,357,163, WO 92/20702 and WO 92/20703).

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Komberg and Baker, DNA Replication, 2nd Ed. (Freeman 1992)). The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and/or nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

In some embodiments, a nucleobase polymer is an polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190.

In some embodiments, a nucleobase polymer is a polynucleotide mimic or oligonucleotide mimic. By “polynucleotide mimic or oligonucleotide mimic” is meant refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g,, Stirchak and Summerton, 1987, J. Org. Chem. 52:4202); amide backbones (see, e.g., Lebreton, 1994, Synlett. February, 1994:137); methylhydroxylamine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoul et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, 1. Chem. Soc. Perkin Trans. 11:555-560; Howarth et al., 1997, I. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Left., 38:4211-4214; Ciapetti et al., 1997, Tetrahedron, 53:1167-1176; Lagriffoule et al., 1997, Chem. Eur. 1.’ 3:912-919; Kumar et al., 2001, Organic Letters 3(9):1269-1272; and the Peptide-Based Nucleic Acid Mimics (PENAMs) of Shah et al. as disclosed in WO 96/04000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500).

In some embodiments, a nucleobase polymer is a chimeric oligonucleotide. By “chimeric oligonucleotide” is meant a nucleobase polymer or oligomer comprising a plurality of different polynucleotides, polynucleotide analogs and polynucleotide mimics. For example a chimeric oligo may comprise a sequence of DNA linked to a sequence of RNA. Other examples of chimeric oligonucleotides include a sequence of DNA linked to a sequence of PNA, and a sequence of RNA linked to a sequence of PNA.

In some embodiments, a polynucleotide (e.g., an amplification primer, a detection polynucleotide) comprises one or more non-nucleobase moieties. Non-limiting examples of non-nucleobase moieties include but are not limited to a ligand, as described above, a “blocking moiety” suitable for inhibiting polymerase extension of the 3′ terminus of a probe when it is hybridized to a target sequence, and moieties suitable for producing a detectable signal. “Detectable moiety,” “detection moiety” or “label” refer to a moiety that, when attached to the disclosed polynucleotides and other compositions, render such compositions detectable or identifiable using known detection systems (e.g., spectroscopic, radioactive, enzymatic, chemical, photochemical, biochemical, immunochemical, chromatographic or electrophoretic systems). Non-limiting examples of labels include isotopic labels (e.g., radioactive or heavy isotopes), magnetic labels; spin labels, electric labels; thermal labels; colored labels (e.g., chromophores), luminescent labels (e.g., fluorescers, chemiluminescers), enzyme labels (e.g., horseradish peroxidase, alkaline phosphatase, luciferase, β-galactosidase) (Ichiki, et al., 1993, J. Immunol. 150(12):540.8-5417; Nolan, et al., 1988, Proc. Natl. Acad. Sci. USA 85(8):2603-2607)), antibody labels, chemically modifiable labels, and mobility modifier labels. In addition, in some embodiments, such labels include components of ligand-binding partner pairs, as described above.

Fluorescent label,” “fluorescent moiety,” and “fluorophore” refer to a molecule that may be detected via its inherent fluorescent properties. Examples of suitable fluorescent labels include, but are not limited to, fluorescein, rhodamine, tetramethylrhodamine, eosin, erythrosin, coumarin, methyl-coumarins, pyrene, Malacite Green, stilbene, Lucifer Yellow, Cascade BlueJ, Texas Red, IAEDANS, EDANS, BODIPY FL, LC Red 640, phycoerythrin, LC Red 705, Oregon green, Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE), FITC, Rhodamine, Texas Red (Pierce, Rockford, Ill.), Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. In some embodiments, suitable fluorescent labels also include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al, 1996, Curr. Biol. 6:178-182; Stauber, 1998, Biotechniques 24(3):462-471;), enhanced yellow fluorescent protein (EYFP; Clontech Laboratories, Inc., Palo Alto, Calif.), and renilla (WO 92/15673; WO 95/07463; WO 98/14605; WO 98/26277; WO 99/49019; U.S. Pat. Nos. 5,292,658, 5,418,155, 5,683,888, 5,741,668, 5,777,079, 5,804,387, 5,874,304, 5,876,995 and No. 5,925,558). Further examples of fluorescent labels are found in Haugland, Handbook of Fluorescent Probes and Research, 9^{th} Edition, Molecule Probes, Inc. Eugene, Oreg. (ISBN 0-9710636-0-5).

In other embodiments, a fluorescent moiety may be an acceptor or donor molecule of a fluorescence energy transfer (FET) or fluorescent resonance energy transfer (FRET) system, which utilize distance-dependent interactions between the excited states of two molecules in which excitation energy is transferred from a donor molecule to an acceptor molecule (see Bustin, 2000, J. Mol. Endocrinol. 25:169-193; WO2004003510). As known in the art, these systems are suitable for detecting or monitoring changes in molecular proximity, including but not limited to, the release of the flap sequence by the 5′-3′ nuclease activity of a polymerase. Therefore, in some embodiments, a flap probe is labeled with donor and acceptor moieties, which provide a detection system suitable for monitoring the release of the flap sequence in real-time. In some embodiments, the transfer of energy from donor to acceptor results in the production of a detectable signal by the acceptor. In another embodiment, the transfer of energy from donor to acceptor results in quenching of the fluorescent signal produced by the donor. Thus, to detect or monitor the release of a flap sequence, the flap sequence and the target specific sequence of a flap probe each comprise a donor or acceptor moiety in energy transfer proximity. Therefore, depending upon the type of donor-acceptor moieties utilized, the release of the flap sequence may be detected or monitored by an increase or decrease in fluorescence signal. In some embodiments, the ligation of ligation probes may be monitored in an analogous fashion. Examples of donor-acceptor pairs suitable for producing a fluorescent signal include but are not limited to fluorescein-tetramethylrhodamine, IAEDANS-fluorescein, EDANS-dabcyl, fluorescein-QSY 7, and fluorescein-QSY 9. Examples of donor-acceptor pairs suitable for quenching a fluorescent signal include but are not limited to FAM-DABCYL, HEX-DABCYL, TET-DABCYL, Cy3-DABCYL, Cy5-DABCYL,, Cy5.5-DABCYL, rhodamine-DABCYL, TAMRA-DABCYL, JOE-DABCYL, ROX-DABCYL, Cascade Blue-DABCYL, Bodipy-DABCYL, FAM-MGB, Vic-MGB, Ned-MGB, ROX-MGB.

In some embodiments, a label is a mobility modifier. “Mobility modifier” refers to a moiety capable of producing a particular mobility in a mobility-dependent analysis technique, such as, electrophoresis (see, e.g., U.S. Pat. Nos. 5,470,705, 5,514,543, 6,395,486 and 6,734,296). Thus, in some embodiments, a mobility modifier can be an electrophoresis mobility modifier. In some embodiments, an electrophoresis mobility modifier is a polynucleotide polymer (e.g., a ligation partner). In some embodiments, an electrophoresis mobility modifier is nonpolynucleotide polymer. Various non-limiting examples of non-polynucleotide electrophoresis mobility modifiers include but are not limited to polyethylene oxide, polyglycolic acid, polylactic acid, polypeptide, oligosaccharide, polyurethane, polyamide, polysulfonamide, polysulfoxide, polyphosphonate, and block copolymers thereof, including polymers composed of units of multiple subunits linked by charged or uncharged linking groups.

The use of detectable moieties in the detection of specific nucleotides at selected positions of a target sequence by the disclosed methods is within the abilities of the skilled artisan. Factors to be considered in selecting the number and types of detectable moieties and their distribution among the various polynucleotides, include but are not limited to, the number of target polynucleotides to be analyzed (e.g., single-plex vs. multiplex analysis), the method selected for detecting the modified products of the detection polynucleotides, the number and types of detectable moieties than may be discriminated, and the extent to which each specific nucleotide is to be discriminated. For example, in some embodiments, flap sequences may comprise detectable moieties. In some embodiments, such as multiplex target sequence analysis, each flap sequence may comprise a detectable moiety that may be discriminated from the detectable moieties of other flap sequences. Therefore, each released flap sequence may be identified by the emission of a unique signal. However, in some embodiments, each flap sequence may comprise the identical detectable moiety. In these embodiments, each released flap sequence may be individually discriminated if, for example, each flap sequence is substantially unique. For example, in embodiments in which each flap sequence differs in length by at least one nucleobase, the individual flap sequence may be conveniently discriminated by capillary electrophoresis. However, in embodiments in which each flap sequence comprises an identical detectable moiety and comprises a sequence of identical length, the individual flap sequences may be discriminated if, for example, the flap sequence does not share statistically significant sequence homology with the other flap sequences. Therefore, in some embodiments, each released flap sequence may be ligated to a unique ligation partner each comprising a distinguishable mobility modifier to form distinguishable ligation amplicons, which also may be individually detected by capillary electrophoresis (e.g., ABI Prism® capillary electrophoresis instruments, Applied Biosystems, Foster City, Calif.). As the skilled artisan will appreciate, these examples of approaches to discriminate individual flap sequence also may be applied to the discrimination of individual ligation products and amplicons.

In various embodiments, the accumulation of product, e.g., nucleotide specific amplicons, a modified detection probe, can be monitored in real-time by carrying out the disclosed methods in the presence of a reporter molecule that generates a detectable signal in proportion to the amount of product present in a reaction. By “reporter molecule” herein is meant a molecule that produces a differential signal when specifically or non-specifically bound to a single-stranded polynucleotide relative to the unbound molecule. Non-limiting examples of reporter molecules include sequence-independent binding agents and sequence-specific binding agents. By “sequence-independent binding” is meant differential binding that is based on structure other than the sequence of a polynucleotide. Therefore, non-limiting examples of structure-specific binding agents include intercalating agents, such as, actinomycin D which fluoresces red when bound to single-stranded polynucleotides and green when bound to double-stranded polynucleotides. By “sequence-specific binding” is meant differential binding based on the sequence of a polynucleotide. Therefore, in some embodiments, a sequence-specific reporter molecule is an oligonucleotide probe. Such oligonucleotide probes include, but are not limited to, hydrolyzable probes (see, e.g., 5′-nuclease probes, (e.g., self-quenching fluorescent probes, e.g., TaqMan® probes), various stem-loop molecular beacons (see, e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA molecular beacons (see, e.g., U.S. Pat. No. 6,355,421), linear PNA beacons (see, e.g., Kubista et al., 2001, SPIE 4264:53-58), non-FRET probes (see, e.g., U.S. Pat. No. 6,150,097) and the various different sunrise primers, scorpion probes, cyclicons (Kandimalla et al., 2000, Bioorg Med. Chem. 8(8):1911-6), peptide nucleic acid (PNA) light-up probes, self-assembled nanoparticle probes (Taton et al., 2000, Science. 289(5485): 1757-60), dual-probe systems, and ferrocene-modified probes described, for example, in U.S. Pat. No. 6,485,901; Mhlanga et al., 2001, Methods. 25:463-471; Whitcombe et al., 1999, Nat. Biotechnol. 17:804-807; Isacsson et al., 2000, Mol Cell Probes. 14:321-328; Svanvik et al., 2000, Anal. Biochem. 281:26-35; Wolffs et al., 2001, Biotechniques. 766:769-771; Tsourkas et al., 2002, Nucleic Acids Res. 30:42084215; Riccelli et al., 2002, Nucleic Acids Res. 30:4088-4093; Zhang et al., 2002, Shanghai. 34:329-332; Maxwell et al., 2002, J Am Chem Soc. 124:9606-9612; Broude et al., 2002, Trends Biotechnol. 20:249-56; Huang et al., 2002, Chem Res Toxicol. 15:118-126; and Yu et al., 2001, J. Am. Chem. Soc. 14:11155-11161) and hydrolyzable “flap” probes, as described above and in U.S. Patent Application Ser. Nos. 60/584,596; 60/584,665; 60/584,621, each filed Jun. 30, 2005.

In some embodiments, the detectable signal is measured at one or more discrete time points or is continuously monitored in real-time. In these embodiments, continuous or discrete monitoring may utilize a reporter molecule comprising a donor-acceptor pair, e.g., fluorophore-quencher pair, as described above. Detection of the fluorescent signal can be performed in any appropriate way based, in part, upon the type of reporter molecule employed (e.g., 5′-nuclease probe vs. a molecular beacon) as known in the art. In some embodiments, the signal may be compared against a control signal or standard curve. Non-limiting examples of existing apparatuses that may be used to monitor the reaction in real-time or take one or more single time point measurements include, Models 7300, 7500, and 7700 Real-Time PCR Systems (Applied Biosystems, Foster City, Calif.); the MyCyler and iCycler Thermal Cyclers (Bio-Rad, Hercules, Calif.); the Mx3000P™ and Mx4000® (Stratagene®, La Jolla, Calif.); the Chromo 4™ Four-Color Real-Time System (MJ Research, Inc., Reno, Nev.); and the LightCycler® 2.0 Instrument (Roche Applied Science, Indianapolis, Ind.).

Also provided are kits for use in practicing the various embodiments of the disclosed methods. Therefore, in some embodiments kits include one or more sets of nucleotide specific amplification primers for producing one more nucleotide specific amplicons and detection polynucleotides suitable for detecting the one or more nucleotide specific amplicons. In some embodiments, the nucleotide specific amplification primers comprise sequences, including but not limited to, one or more universal sequences and/or code sequences, which in some embodiments provide hybridization targets for the detection polynucleotides. In some embodiments, the detection polynucleotides comprise one or more primers and flap probes. In some embodiments, the detection polynucleotides comprises two or more ligation probes. In some embodiments, a kit may further comprise a polymerase suitable to amplify a target sequence and/or a polymerase having 5′-3′ nuclease activity. In various embodiments, kits may further comprise moieties suitable for producing a detectable signal or reporter molecules suitable for monitoring, for example, the accumulation of the nucleotide specific target sequence or modification of a detection polynucleotide, as described above.

The following examples are offered by way of illustration and not by way of limitation. All literature and similar materials cited in this application, including but not limited to, patents, patent applications, articles, books, and treatises, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### 6. EXAMPLES

### Example 1

### Single Nucleotide Polymorphism Analysis of CEPH GDNA

CEPH gDNA (Center for the Study of Human Polymorphism) was analyzed in a multiplex reaction for 12 single nucleotide polymorphisms (SNPs). Each SNP is identified according to the Celera Genomic Database (Applera Corporation) designation in the name of the allele specific primers (ASO-1 and ASO-2) shown in Tables 4 and 5, and the locus specific primers (LS-RP) shown in Table 1. Therefore to amplifying SNP CV3200530, the allele specific forward primers used (ASO-1 and ASO-2, respectively) are CV3200530-CODE1G2, CV3200530-CODE1A1. The reverse primer is UR-RPCV3200530. The LS-RP primers incorporate a universal reverse primer sequence into the amplicons and provide a site for linear amplification using the universal reverse primer (Table 2). Following amplification, the linear amplicons are hybridized to a ligation partner (Table 3) and either FAM-GA or VIC-AG (Table 2) depending on whether an ASO-1 or an ASO-2 primer produced the nucleotide specific amplicon.

Various amounts of gDNA (5 ng, 50 ng) were amplified in a reaction comprising 1×ABI Master Mix (Applied Biosystems, Foster City, Calif.), 1 μM universal reverse primer, nM ASO-1 and ASO-2 primers, and 5 nM LS-RP primers. The amplification reaction was thermocycled 45 times (95° C. for 15 sec.-65° C. for 1 min.). An aliquot of the amplification reaction was used as a template for the ligation of FAM-GA or VIC-GA to one of the ligation partners. The reaction comprised 1×ABI Ligase Buffer (Applied Biosystems, Foster City, Calif.), 20 nM ligation partners, 20 nM FAM-GA and VIC-GA, 10 U AK16D Ligase (Thermus sp. isolate AK16D). The reaction was thermocycled 30 times (94° C. for 5 sec.-60° C. for 1 min.). An aliquot of the ligase reaction was mixed with 9.5 μl DI Formamide and analyzed by CE.

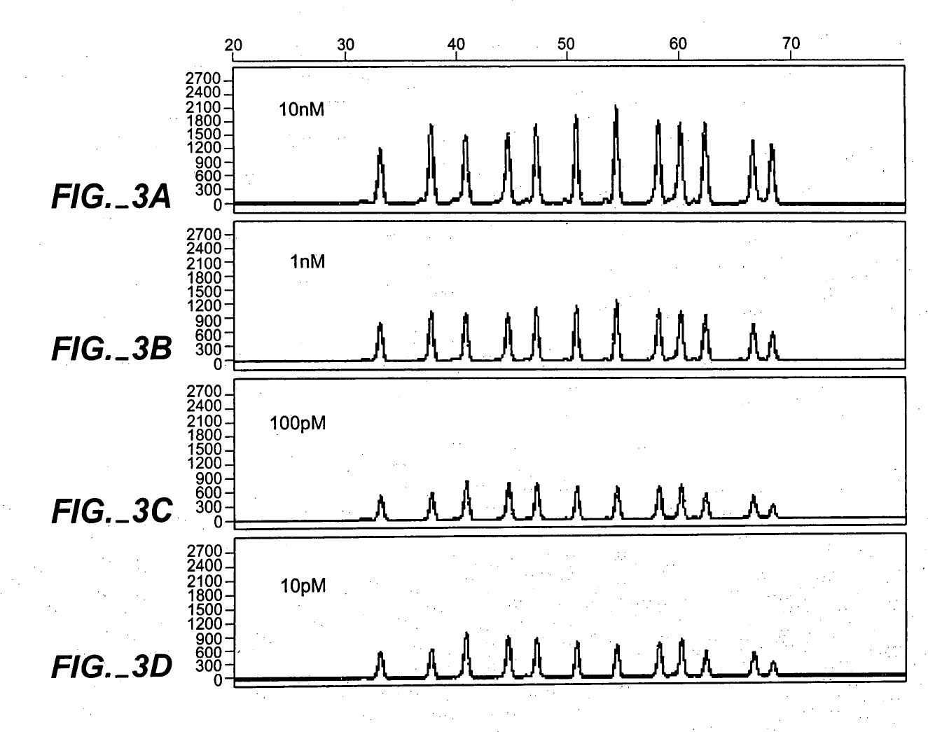

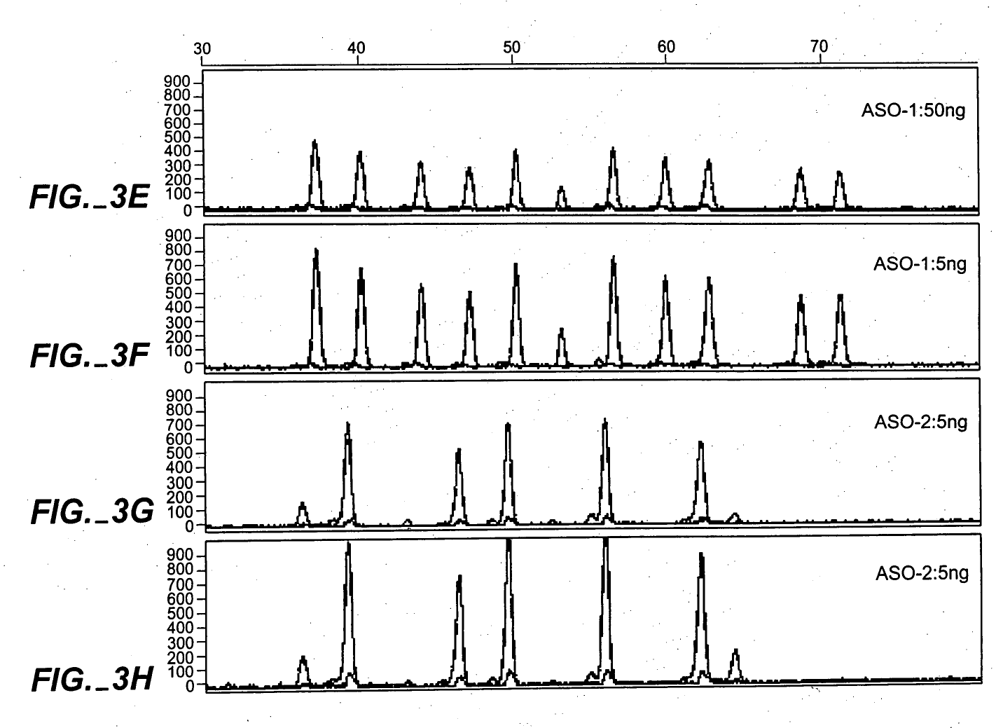

The results are shown in FIGS. 3E-G for the various amounts of gDNA analyzed. Each peak represents a SNP detected in the gDNA. Differences in the peaks seen in FIGS. 3E-F (ASO-1) as compared to FIGS. 3G-H (ASO-2) indicate the gDNA only contains one of the SNPs that could be detected by either the ASO-1 or ASO-2 primers. For example, the two peaks are the far right of FIGS. 3E-F are absent from FIGS. 3G-H. Therefore, the SNP detectable by ASO-2 is absent from the gDNA.

FIG. 3A is the result of capillary electrophoresis of control ligation amplicons that were used to examine the sensitivity of the detection system.

TABLE 1

| | |
|---|---|
| Locus Specific Reverse Primers | SEQ ID NO: |

| | | |
|---|---|---|
| UR-RPCV2617626 | 5′-ACCGACTCCAGCTCCCGAACGGAAGGAAACCTGAGTGCACAGTT | SEQ ID NO:01 |
| UR-RPCV2617615 | 5′-ACCGACTCCAGCTCCCGAACTGCAGTAAGTCTTTTTTCTGGGCT | SEQ ID NO:02 |
| UR-RPCV7452444 | 5′-ACCGACTCCAGCTCCCGAACTGTAAAACCCATACCTCTAAGAAAGGAT | SEQ ID NO:03 |
| UR-RPCV57604 | 5′-ACCGACTCCAGCTCCCGAACGTGTGATTAAACATGTGGTTGGTGC | SEQ ID NO:04 |
| UR-RPCV11625923 | 5′-ACCGACTCCAGCTCCCGAACCTAACAGCGAGAGGACGGGTTA | SEQ ID NO:05 |
| UR-RPCV3200530 | 5′-ACCGACTCCAGCTCCCGAACGGGAAATGAATATGGTAAAGACACAACTA | SEQ ID NO:06 |
| UR-RPCV3290005 | 5′-ACCGACTCCAGCTCCCGAACCAAGGACCAGATGACTGAGATGTCA | SEQ ID NO:07 |
| UR-RPCV2617621 | 5′-ACCGACTCCAGCTCCCGAACATGACTGACTTCTCATTTGGGATCA | SEQ ID NO:08 |
| UR-RPCV1987171 | 5′-ACCGACTCCAGCTCCCGAACATCACTAAAGAAGGTAAGACTCAGGAACAA | SEQ ID NO:09 |
| UR-RPCV8727884 | 5′-ACCGACTCCAGCTCCCGAACCGCACTAAGTGGATACTCTGTTGCTGT | SEQ ID NO:10 |
| UR-RPCV12123528 | 5′-ACCGACTCCAGCTCCCGAACCAGAAAAATGAACAAAGATAAGTACATATACGT | SEQ ID NO:11 |
| UR-RPCV3194764 | 5′-ACCGACTCCAGCTCCCGAACCCGGTAGAGAGCCCTGGGAGTC | SEQ ID NO:12 |

TABLE 2

| | | |
|---|---|---|
| Univiversal | 5′-ACCGACTCCAGCTCCCGAAC | SEQ ID NO:13 |
| Reverse (UR) | | |
| FAM-GA | 5′-FAM-TGTGTCGTGGAGTCGG | SEQ ID NO:14 |
| | CAAGA | |
| VIC-AG | 5′-VIC-GTGTCGTGGAGTCGGC | SEQ ID NO:15 |
| | AAAG | |

TABLE 3

Ligation Partners

| | | |
|---|---|---|
| LDR2-CODE1-43 | 5′-pAGCGAGCGGGAACAGGCCAATT-C6*-NH2 | SEQ ID NO:16 |
| LDR2-CODE1-44 | 5′-pGGAACACCACGCAGCGCAGGTTTT-C6-NH2 | SEQ ID NO:17 |
| LDR2-CODE1-45 | 5′-pGCAGTGCTCACCGTCCGCGATTTTTT-C6-NH2 | SEQ ID NO:18 |
| LDR2-CODE1-46 | 5′-pCGGAGTGGCACCAGCGGGAATTTTTTTT-C6-NH2 | SEQ ID NO:19 |
| LDR2-CODE1-47 | 5′-pGCAGCAGGCCAAAGCGAGCGTTTTTTTTTT-C6-NH2 | SEQ ID NO:20 |
| LDR2-CODE1-48 | 5′-pGTCCGAGCCCTCACGCAGCGTTTTTTTTTTTT-C6-NH2 | SEQ ID NO:21 |
| LDR2-CODE1-49 | 5′-pGCAGGACGACGCGGGTGGAATTTTTTTTTTTTTT-C6-NH2 | SEQ ID NO:22 |
| LDR2-CODE1-50 | 5′-pTGGCGGTCTGCTGAGCGGTCTTTTTTTTTTTTTTTT-C6-NH2 | SEQ ID NO:23 |
| LDR2-CODE1-51 | 5′-pGTGGGTCCCGGAAGCGTGCTTTTTTTTTTTTTTTTTTT-C6-NH2 | SEQ ID NO:24 |
| LDR2-CODE1-52 | 5′-pGCCTCGAGCCAACACCGCCTTTTTTTTTTTTTTTTTTTTT-C6-NH2 | SEQ ID NO:25 |
| LDR2-CODE1-53 | 5′-pTGGCCGGACAGGAGACACGCTTTTTTTTTTTTTTTTTTTTTT-C6-NH2 | SEQ ID NO:26 |

*C6 refers to an six-carbon alkyl chain.

TABLE 4

Allele Specific Primers (ASO-1)

| | | |
|---|---|---|
| CV3200530CODE1G2 | 5′C6-GTGTCGTGGAGTCGGCAAAGAGCGAGCGGGAACAGGCCAACTGAGCAACATAATCAATATTCACG | SEQ ID NO:27 |
| CV3290005CODE4C11 | 5′C6-GTGTCGTGGAGTCGGCAAAGCGGAGTGGCACCAGCGGGAAGGAAGGGAAGAGATOGGATTC | SEQ ID NO:28 |
| CV8727884CODE5G14 | 5′C6-GTGTCGTGGAGTCGGCAAAGGCAGCAGGCCAAAGCGAGCGTGCGGCCAGTTGATCCG | SEQ ID NO:29 |
| CVCV3194764CODE7C20 | 5′C6-GTGTCGTGGAGTCGGCAAAGGCAGGACGACGCGGGTGGAAGCGGTCTCCCCACCTCAC | SEQ ID NO:30 |
| CV57604CODE3G8 | 5′C6-GTGTCGTGGAGTCGGCAAAGGCAGTGCTCACCGTCCGCGAAATTCCCATTTTGGTTTCCATG | SEQ ID NO:31 |
| CV2617615CODE10C29 | 5′C6-GTGTCGTGGAGTCGGCAAAGGCCTCGAGCCAACACCGCCTCAATACATACAAATAGGACCAGAA | SEQ ID NO:32 |
| | GC | |
| CV2617626CODE12C35 | 5′C6-GTGTCGTGGAGTCGGCAAAGGCCTGCCTTCACGAGCCCAATTTAATCTCAGACTGGOTGTTACAC | SEQ ID NO:33 |
| CV1987171CODE2G5 | 5′C6-GTGTCGTGGAGTCGGCAAAGGGAACACCACGCAGCGCAGGTCTCACATGTGGGGCCATG | SEQ ID NO:34 |
| CV12123528CODE6C17 | 5′C6-GTGTCGTGGAGTCGGCAAAGGTCCGAGCCCTCACGCAGCGAATTCCGTACATTTTTGTTAGTTGC | SEQ ID NO:35 |
| CV11625923CODE9C26 | 5′C6-GTGTCGTGGAGTCGGCAAAGGTGGGTCCCGOAAGCGTGCTCTGTTTGTGCCTCTCTCTGTTAC | SEQ ID NO:36 |
| CV2617621CODE11G32 | 5′C6-GTGTCGTGGAGTCGGCAAAGTGGCCGGACAGGAGACACGCAACTCAGAGGTTACCAAGCCTAG | SEQ ID NO:37 |
| CV7452444CODE8G23 | 5′C6-GTGTCGTGGAGTCGGCAAAGTGGCGGTCTGCTGAGCGGTCAGGGGTCAGGATTTTTCTGAG | SEQ ID NO:38 |

TABLE 5

Allele Specific Primers (ASO-2)

| | | |
|---|---|---|
| CV3200530CODE1A1 | 5′C6-GTGTCGTGGAGTCGGCAAGAAGCGAGCGGGAACAGGCCAACTGAGCAACATAATCAATATTCACAC | SEQ ID NO:39 |
| CV3290005CODE4T10 | 5′C6-GTGTCGTGGAGTCGGCAAGACGGAGTGGCACCAGCGGGAAGGAAGGGAAGAGATGGGATTT | SEQ ID NO:40 |
| CV8727884CODE5A13 | 5′C6-GTGTCGTGGAGTCGGCAAGAGCAGCAGGCCAAAGCGAGCGTGCGGCCAGTTGATCCA | SEQ ID NO:41 |
| CV3194764CODE7T1 | 5′C6-GTGTCGTGGAGTCGGCAAGAGCAGGACGACGCGGGTGGAAGCGGTCTCCCCACCTCAT | SEQ ID NO:42 |
| CV57604CODE3A7 | 5′C6-GTGTCGTGGAGTCGGCAAGAGCAGTGCTCACCGTCCGCGAAATTCCCATTTTGGTTTCCATA | SEQ ID NO:43 |
| CV2617615CODE10A28 | 5′C6-GTGTCGTGGAGTCGGCAAGAGCCTCGAGCCAACACCGCCTCAATACATACAAATAGGACCAGAAGA | SEQ ID NO:44 |
| CV2617626CODE12A34 | 5′C6-GTGTCGTGGAGTCGGCAAGAGCCTGCCTTCACGAGCCCAATTTAATCTCAGACTGGGTGTTACAA | SEQ ID NO:45 |
| CV1987171CODE2C4 | 5′C6-GTGTCGTGGAGTCGGCAAGAGGAACACCACGCAGCGCAGGTCTCACATGTGGGGCCATC | SEQ ID NO:46 |
| CV12123528CODE6A6 | 5′C6-GTGTCGTGGAGTCGGCAAGAGTCCGAGCCCTCACGCAGCGAATTCCGTACATTTTTGTTAGTTGA | SEQ ID NO:47 |
| CV11625923CODE9T25 | 5′C6-GTGTCGTGGAGTCGGCAAGAGTGGGTCCCGGAAGCGTGCTCTGTTTGTGCCTCTCTCTGTTAT | SEQ ID NO:48 |
| CV2617621CODE11A31 | 5′C6-GTGTCGTGGAGTCGGCAAGATGGCCGGACAGGAGACACGCAACTCAGAGGTTACCAAGCCTAA | SEQ ID NO:49 |
| CV7452444CODE8A22 | 5′C6-GTGTCGTGGAGTCGGCAAGATGGCGGTCTGCTGAGCGGTCAGGGGTCAGGATTTTTCTGAA | SEQ ID NO:50 |

What is claimed is:

1. A method of identifying a specific nucleotide at a selected position of a target sequence, comprising:
a) amplifying a target sequence comprising a specific nucleotide at a selected position with a polymerase having 5′-3′ nuclease activity, and forward and reverse amplification primers to produce an amplicon, wherein said forward amplification primer comprises a 3′ sequence that is suitable to specifically amplify said target sequence, a code sequence and a universal sequence, and wherein said amplicon comprises a forward strand and a reverse strand, wherein said forward strand comprises said universal sequence at its 5′ terminus, said code sequence, and a 3′ sequence complementary to said target sequence, and said reverse strand comprises a sequence complementary to said forward strand;
b) hybridizing to said reverse strand a detection primer comprising said universal sequence and a flap probe comprising said code sequence, under conditions suitable for form a substrate for said 5′-3′ nuclease activity and for said nuclease activity to release the flap sequence from said probe; and
c) detecting said released flap sequence, whereby said specific nucleotide is identified.

2. The method according to claim 1, wherein said released flap sequence is detected by capillary electrophoresis.

3. The method according to claim 1, wherein said released flap sequence comprises a fluorophore.

4. The method according to claim 1, further comprising hybridizing said released flap sequence to a template in the presence of a ligation partner and a ligase, and under conditions suitable for said ligase to join said flap sequence and ligation partner.

5. The method according to claim 4, wherein said ligation partner comprises an electrophoresis mobility modifier.

6. The method according to claim 4, wherein said conditions suitable for joining said flap and ligation partner comprise multiple rounds of thermocycling.

7. The method according to claim 1, wherein the concentration of said forward and reverse primers are about 5 nM to about 20 nM.

8. The method according to claim 1, where said flap probe comprises a labeling system suitable for monitoring the release of said flap sequence.

9. The method according to claim 1, wherein said detection primer and flap probe are hybridized to said reverse strand under conditions suitable to extend said detection primer by the action of said polymerase.

10. The method according to claim 9, wherein said conditions suitable to extend said detection primer comprise multiple rounds of thermocycling.

11. A method of identifying a plurality of specific nucleotides at selected positions of a plurality of target sequences, comprising:
a) amplifying a plurality of target sequences comprising specific nucleotides at selected positions with a polymerase having 5′-3′ nuclease activity, and a plurality of forward and reverse amplification primers to produce a plurality of amplicons, wherein said forward amplification primers each comprise a ₃′ sequence that is suitable to specifically amplify one of said target sequences, one of a plurality of code sequences and a universal sequence, and wherein each of said amplicons comprises forward and reverse strands, wherein said forward strands comprise said universal sequence at their 5′ termini, one of said plurality of code sequences, and a 3′ sequence complementary to one of said target sequences, and said reverse strands comprise sequences complementary to said forward strands;
b) hybridizing to said reverse strands a detection primer comprising said universal sequence and one of a plurality of flap probes each comprising one of said code sequences, under conditions suitable to form a substrate for said nuclease activity and for said nuclease activity to release the flap sequences from said probes; and
c) detecting each of said released flap sequences, whereby said plurality of specific nucleotides are identified.

12. The method according to claim 11, wherein said released flap sequences are detected by capillary electrophoresis.

13. The method according to claim 11, wherein said released flap sequences comprise a fluorophore.

14. The method according to claim 11, further comprising hybridizing said released flap sequences to a plurality of template in the presence of a plurality of ligation partners and a ligase, and under conditions suitable for said ligase to join each of said flap sequences and ligation partners.

15. The method according to claim 14, wherein each of said ligation amplicons comprise a distinguishable electrophoresis mobility modifier.

16. The method according to claim 14, wherein said conditions suitable for joining said flap sequence and ligation partner comprise multiple rounds of thermocycling.

17. The method according to claim 11, wherein the concentrations of said forward and reverse primers are about 5 nM to about 20 nM.

18. The method according to claim 11, where each flap probe comprises a labeling system suitable for monitoring the release of said flap sequences.

19. The method according to claim 11, wherein said detection primers and flap probes are hybridized to said reverse strand under conditions suitable to extend said detection primers by the action of said polymerase.

20. The method according to claim 19, wherein said conditions suitable to extend said detection primers comprise multiple rounds of thermocycling.

21. A method of identifying a specific nucleotide at a selected position of a target sequence, comprising:
a) amplifying a target sequence comprising a specific nucleotide at a selected position with a polymerase, and forward and reverse amplification primers to produce an amplicon, wherein said forward amplification primer comprises a 3′ sequence that is suitable to specifically amplify said target sequence, a code sequence and a universal sequence, and wherein said amplicon comprises forward and reverse strands, wherein said forward strand comprises said universal sequence at its 5′ terminus, said code sequence, and a 3′ sequence complementary to target sequence, and said reverse strand comprises a sequence complementary to said forward strand;
b) hybridizing to said reverse strand a first ligation probe comprising said universal sequence and a second ligation probe comprising said code sequence in the presence of a ligase, and under conditions suitable for said ligase to join said first and second ligation sequences to form a ligation amplicon; and
c) detecting said ligation amplicon, whereby said specific nucleotide is identified.

22. The method according to claim 21, wherein said ligation amplicon is detected by capillary electrophoresis.

23. The method according to claim 21, wherein one of said ligation probes comprises a fluorophore.

24. The method according to claim 21, wherein one of said ligation probes comprises an electrophoresis mobility modifier.

25. The method according to claim 21, wherein said conditions suitable for joining said ligation probes further comprise multiple rounds of thermocycling.

26. The method according to claim 21, wherein the concentration of said forward and reverse primers are about 5 nM to about 20 nM.

27. A method of identifying a plurality of specific nucleotides at selected positions of a plurality of target sequences, comprising:
a) amplifying a plurality of target sequences comprising specific nucleotides at selected positions with a polymerase, and a plurality of forward and reverse amplification primers to produce a plurality of amplicons, wherein said forward amplification primers each comprise a 3′ sequence that is suitable to specifically amplify one of said target sequences, one of a plurality of code sequences and a universal sequence, and wherein each of said amplicons comprises forward and reverse strands, wherein said forward strands comprise said universal sequence at their 5′ termini, one of said plurality of code sequences, and a 3′ sequence complementary to one of said target sequences, and said reverse strands comprise sequences complementary to said forward strands;
b) hybridizing to each reverse strand a first ligation probe comprising said universal sequence and one of a plurality of second ligation probes each comprising one of said code sequences in the presence of a ligase, and under conditions suitable for said ligase to join one of said first ligation probes to one of said second ligation probes to form a plurality of ligation amplicons; and
c) detecting said plurality of ligation amplicons, whereby said plurality of specific nucleotides are identified.

28. The method according to claim 27, wherein said plurality of ligation amplicons are detected by capillary electrophoresis.

29. The method according to claim 27, wherein said first or second ligation probes comprise a fluorophore.

30. The method according to claim 27, wherein said first or second ligation probes each comprise an distinguishable electrophoresis mobility modifier.

31. The method according to claim 27, wherein said conditions suitable for joining said ligation probes comprise multiple rounds of thermocycling.

32. The method according to claim 27, wherein the concentrations of said forward and reverse primers are about 5 nM to about 20 nM.

33. A method of identifying a specific nucleotide at a selected position of a target sequence, comprising:
a) amplifying a target sequence comprising a specific nucleotide at a selected position with a polymerase having 5′-3′ nuclease activity, and at least one amplification primer to produce an amplicon, wherein at least one of primers comprise a 3′ sequence that is suitable to specifically amplify said target sequence, a code sequence and a universal sequence;
b) hybridizing to said amplicon a detection primer and a flap probe, wherein at least one of said detection primers or said flap probes hybridizes to said code sequence or a sequence complementary thereto, and said flap probe hybridizes 3′ relative to said detection primer, under conditions suitable for said 5′-3′ nuclease activity to release the flap sequence from said probe; and
c) detecting said released flap sequence, whereby said specific nucleotide is identified.

34. A method of identifying a specific nucleotide at a selected position of a target sequence, comprising:
a) amplifying a target sequence comprising a specific nucleotide at a selected position with a polymerase, and at least one amplification primer to produce an amplicon, wherein at least one of said primers comprises a 3′ sequence that is suitable to specifically amplify said target sequence, a code sequence and a universal sequence;
b) hybridizing to said amplicon first and second ligation probes in the presence of a ligase, wherein at least one of said ligation probes hybridizes to said code sequence or a sequence complementary thereto, and the other of said ligation probes hybridizes at a position suitable to form a substrate for said ligase, under conditions suitable for said ligase to join said ligation probes to form a ligation amplicon; and
c) detecting said ligation amplicon, whereby said specific nucleotide is identified.


SEQUENCE LISTING (50 sequences)

SEQ ID NO: 1 - DNA, 44 bp, Artificial Sequence
  Description of Artificial Sequence Synthetic primer
accgactccagctcccgaacggaaggaaacctgagtgcacagtt

SEQ ID NO: 2 - DNA, 44 bp, Artificial Sequence
  Description of Artificial Sequence Synthetic primer
accgactccagctcccgaactgcagtaagtcttttttctgggct

SEQ ID NO: 3 - DNA, 48 bp, Artificial Sequence
  Description of Artificial Sequence Synthetic primer
accgactccagctcccgaactgtaaaacccatacctctaagaaaggat

SEQ ID NO: 4 - DNA, 45 bp, Artificial Sequence
  Description of Artificial Sequence Synthetic primer
accgactccagctcccgaacgtgtgattaaacatgtggttggtgc

SEQ ID NO: 5 - DNA, 42 bp, Artificial Sequence
  Description of Artificial Sequence Synthetic primer
accgactccagctcccgaacctaacagcgagaggacgggtta

SEQ ID NO: 6 - DNA, 49 bp, Artificial Sequence
  Description of Artificial Sequence Synthetic primer
accgactccagctcccgaacgggaaatgaatatggtaaagacacaacta

SEQ ID NO: 7 - DNA, 45 bp, Artificial Sequence
  Description of Artificial Sequence Synthetic primer
accgactccagctcccgaaccaaggaccagatgactgagatgtca

SEQ ID NO: 8 - DNA, 45 bp, Artificial Sequence
  Description of Artificial Sequence Synthetic primer
accgactccagctcccgaacatgactgacttctcatttgggatca

SEQ ID NO: 9 - DNA, 50 bp, Artificial Sequence
  Description of Artificial Sequence Synthetic primer
accgactccagctcccgaacatcactaaagaaggtaagactcaggaacaa

SEQ ID NO: 10 - DNA, 47 bp, Artificial Sequence
  Description of Artificial Sequence Synthetic primer
accgactccagctcccgaaccgcactaagtggatactctgttgctgt

SEQ ID NO: 11 - DNA, 53 bp, Artificial Sequence
  Description of Artificial Sequence Synthetic primer
accgactccagctcccgaaccagaaaaatgaacaaagataagtacatatacgt

SEQ ID NO: 12 - DNA, 42 bp, Artificial Sequence
  Description of Artificial Sequence Synthetic primer
accgactccagctcccgaacccggtagagagccctgggagtc

SEQ ID NO: 13 - DNA, 20 bp, Artificial Sequence
  Description of Artificial Sequence Synthetic primer
accgactccagctcccgaac

SEQ ID NO: 14 - DNA, 21 bp, Artificial Sequence
  Description of Artificial Sequence Synthetic oligonucleotide
tgtgtcgtggagtcggcaaga

SEQ ID NO: 15 - DNA, 20 bp, Artificial Sequence
  Description of Artificial Sequence Synthetic oligonucleotide
gtgtcgtggagtcggcaaag

SEQ ID NO: 16 - DNA, 22 bp, Artificial Sequence
  Description of Artificial Sequence Synthetic oligonucleotide
agcgagcgggaacaggccaatt

SEQ ID NO: 17 - DNA, 24 bp, Artificial Sequence
  Description of Artificial Sequence Synthetic oligonucleotide
ggaacaccacgcagcgcaggtttt

SEQ ID NO: 18 - DNA, 26 bp, Artificial Sequence
  Description of Artificial Sequence Synthetic oligonucleotide
gcagtgctcaccgtccgcgatttttt

SEQ ID NO: 19 - DNA, 28 bp, Artificial Sequence
  Description of Artificial Sequence Synthetic oligonucleotide
cggagtggcaccagcgggaatttttttt

SEQ ID NO: 20 - DNA, 30 bp, Artificial Sequence
  Description of Artificial Sequence Synthetic oligonucleotide
gcagcaggccaaagcgagcgtttttttttt

SEQ ID NO: 21 - DNA, 32 bp, Artificial Sequence
  Description of Artificial Sequence Synthetic oligonucleotide
gtccgagccctcacgcagcgtttttttttttt

SEQ ID NO: 22 - DNA, 34 bp, Artificial Sequence
  Description of Artificial Sequence Synthetic oligonucleotide
gcaggacgacgcgggtggaatttttttttttttt

SEQ ID NO: 23 - DNA, 36 bp, Artificial Sequence
  Description of Artificial Sequence Synthetic oligonucleotide
tggcggtctgctgagcggtctttttttttttttttt

SEQ ID NO: 24 - DNA, 38 bp, Artificial Sequence
  Description of Artificial Sequence Synthetic oligonucleotide
gtgggtcccggaagcgtgcttttttttttttttttttt

SEQ ID NO: 25 - DNA, 40 bp, Artificial Sequence
  Description of Artificial Sequence Synthetic oligonucleotide
gcctcgagccaacaccgccttttttttttttttttttttt

SEQ ID NO: 26 - DNA, 42 bp, Artificial Sequence
  Description of Artificial Sequence Synthetic oligonucleotide
tggccggacaggagacacgctttttttttttttttttttttt

SEQ ID NO: 27 - DNA, 65 bp, Artificial Sequence
  Description of Artificial Sequence Synthetic primer
gtgtcgtggagtcggcaaagagcgagcgggaacaggccaactgagcaacataatcaatattcacg

SEQ ID NO: 28 - DNA, 61 bp, Artificial Sequence
  Description of Artificial Sequence Synthetic primer
gtgtcgtggagtcggcaaagcggagtggcaccagcgggaaggaagggaagagatgggattc

SEQ ID NO: 29 - DNA, 57 bp, Artificial Sequence
  Description of Artificial Sequence Synthetic primer
gtgtcgtggagtcggcaaaggcagcaggccaaagcgagcgtgcggccagttgatccg

SEQ ID NO: 30 - DNA, 58 bp, Artificial Sequence
  Description of Artificial Sequence Synthetic primer
gtgtcgtggagtcggcaaaggcaggacgacgcgggtggaagcggtctccccacctcac

SEQ ID NO: 31 - DNA, 62 bp, Artificial Sequence
  Description of Artificial Sequence Synthetic primer
gtgtcgtggagtcggcaaaggcagtgctcaccgtccgcgaaattcccattttggtttccatg

SEQ ID NO: 32 - DNA, 66 bp, Artificial Sequence
  Description of Artificial Sequence Synthetic primer
gtgtcgtggagtcggcaaaggcctcgagccaacaccgcctcaatacatacaaataggaccagaagc

SEQ ID NO: 33 - DNA, 65 bp, Artificial Sequence
  Description of Artificial Sequence Synthetic primer
gtgtcgtggagtcggcaaaggcctgccttcacgagcccaatttaatctcagactgggtgttacac

SEQ ID NO: 34 - DNA, 59 bp, Artificial Sequence
  Description of Artificial Sequence Synthetic primer
gtgtcgtggagtcggcaaagggaacaccacgcagcgcaggtctcacatgtggggccatg

SEQ ID NO: 35 - DNA, 65 bp, Artificial Sequence
  Description of Artificial Sequence Synthetic primer
gtgtcgtggagtcggcaaaggtccgagccctcacgcagcgaattccgtacatttttgttagttgc

SEQ ID NO: 36 - DNA, 63 bp, Artificial Sequence
  Description of Artificial Sequence Synthetic primer
gtgtcgtggagtcggcaaaggtgggtcccggaagcgtgctctgtttgtgcctctctctgttac

SEQ ID NO: 37 - DNA, 63 bp, Artificial Sequence
  Description of Artificial Sequence Synthetic primer
gtgtcgtggagtcggcaaagtggccggacaggagacacgcaactcagaggttaccaagcctag

SEQ ID NO: 38 - DNA, 61 bp, Artificial Sequence
  Description of Artificial Sequence Synthetic primer
gtgtcgtggagtcggcaaagtggcggtctgctgagcggtcaggggtcaggatttttctgag

SEQ ID NO: 39 - DNA, 66 bp, Artificial Sequence
  Description of Artificial Sequence Synthetic primer
gtgtcgtggagtcggcaagaagcgagcgggaacaggccaactgagcaacataatcaatattcacac

SEQ ID NO: 40 - DNA, 61 bp, Artificial Sequence
  Description of Artificial Sequence Synthetic primer
gtgtcgtggagtcggcaagacggagtggcaccagcgggaaggaagggaagagatgggattt

SEQ ID NO: 41 - DNA, 57 bp, Artificial Sequence
  Description of Artificial Sequence Synthetic primer
gtgtcgtggagtcggcaagagcagcaggccaaagcgagcgtgcggccagttgatcca

SEQ ID NO: 42 - DNA, 58 bp, Artificial Sequence
  Description of Artificial Sequence Synthetic primer
gtgtcgtggagtcggcaagagcaggacgacgcgggtggaagcggtctccccacctcat

SEQ ID NO: 43 - DNA, 62 bp, Artificial Sequence
  Description of Artificial Sequence Synthetic primer
gtgtcgtggagtcggcaagagcagtgctcaccgtccgcgaaattcccattttggtttccata

SEQ ID NO: 44 - DNA, 66 bp, Artificial Sequence
  Description of Artificial Sequence Synthetic primer
gtgtcgtggagtcggcaagagcctcgagccaacaccgcctcaatacatacaaataggaccagaaga

SEQ ID NO: 45 - DNA, 65 bp, Artificial Sequence
  Description of Artificial Sequence Synthetic primer
gtgtcgtggagtcggcaagagcctgccttcacgagcccaatttaatctcagactgggtgttacaa

SEQ ID NO: 46 - DNA, 59 bp, Artificial Sequence
  Description of Artificial Sequence Synthetic primer
gtgtcgtggagtcggcaagaggaacaccacgcagcgcaggtctcacatgtggggccatc

SEQ ID NO: 47 - DNA, 65 bp, Artificial Sequence
  Description of Artificial Sequence Synthetic primer
gtgtcgtggagtcggcaagagtccgagccctcacgcagcgaattccgtacatttttgttagttga

SEQ ID NO: 48 - DNA, 63 bp, Artificial Sequence
  Description of Artificial Sequence Synthetic primer
gtgtcgtggagtcggcaagagtgggtcccggaagcgtgctctgtttgtgcctctctctgttat

SEQ ID NO: 49 - DNA, 63 bp, Artificial Sequence
  Description of Artificial Sequence Synthetic primer
gtgtcgtggagtcggcaagatggccggacaggagacacgcaactcagaggttaccaagcctaa

SEQ ID NO: 50 - DNA, 61 bp, Artificial Sequence
  Description of Artificial Sequence Synthetic primer
gtgtcgtggagtcggcaagatggcggtctgctgagcggtcaggggtcaggatttttctgaa